SEQUENCE LISTING (5 sequences)

SEQ ID NO: 1 - protein, 107 aa, Artificial sequence
  Synthesized
DIVMTQSQKFMSTSVGDRVSITCKASQNVRTAVAWYQQKPGQSPKALIYLASNRHTGVPDRFTGSGSGTDFTLTISNVQSEDLADYFCLQHWNYPLTFGAGTKLELK

SEQ ID NO: 2 - protein, 119 aa, Artificial sequence
  Synthesized
EVQLQESGGGLVQPKGSLKLSCAASGFTFNTHAMNWVRQAPGKGLEWVARIRSKSNNYATYYADSVKDRFTISRDDSQSMLYLQMNNLKTEDTAMYYCVGDGYYGFTYWGQGTLVTVSA

SEQ ID NO: 3 - DNA, 321 bp, Artificial sequence
  Synthesized
gacattgtgatgacccagtctcaaaaattcatgtccacatcagtaggagacagggtcagcatcacctgcaaggccagtcagaatgttcgtactgctgtagcctggtatcaacagaaaccagggcagtctcctaaagcactgatttacttggcatccaaccggcacactggagtccctgatcgcttcacaggcagtggatctgggacagatttcactctcaccattagcaatgtgcaatctgaagacctggcagattatttctgtctgcaacattggaattatcctctcacgttcggtgctgggaccaagttggagctgaaa

SEQ ID NO: 4 - DNA, 357 bp, Artificial sequence
  Synthesized
gaggtgcagcttcaggagtcaggtggaggattggtgcagcctaaagggtccttgaaactctcatgtgcagcctctggattcaccttcaatacccatgccatgaactgggtccgccaggctccaggaaagggtttggaatgggttgctcgtataagaagtaaaagtaataattatgcaacatattatgccgattcagtgaaagacaggttcaccatctccagagatgattcacaaagcatgctctatctgcaaatgaacaacttgaaaactgaggacacagccatgtattactgtgtgggggatggttactacgggtttacttattggggccaagggactctggtcactgtctctgca

SEQ ID NO: 5 - protein, 15 aa, Artificial sequence
  Synthesized
QLPEDAFKNFPFLEE

(19) United States
(12) Patent Application Publication
(10) Pub. No.: US 2024/0101665 A1
(43) Pub. Date: Mar. 28, 2024

(54) MONOCLONAL ANTIBODY AGAINST HUMAN GPR48 AND APPLICATION THEREOF

(71) Applicant: INSTITUTE OF ZOOLOGY, CHINESE ACADEMY OF SCIENCES, Beijing (CN)

(72) Inventors: Quan CHEN, Beijing (CN); Lei DU, Beijing (CN); Qi CHENG, Beijing (CN); Hao ZHENG, Beijing (CN); Jun WANG, Beichen West Road 1#5 (CN); Xiaohui WANG, Beijing (CN); Lei LIU, Beijing (CN)

(21) Appl. No.: 18/263,883

(22) PCT Filed: Jan. 28, 2022
(86) PCT No.: PCT/CN2022/074456

§ 371 Date: Aug. 1, 2023

(30) Foreign Application Priority Data
Feb. 3, 2021 (CN) 20211047974.2

(51) Int. Cl. C07K 16/28; A61P 35/00; C07K 16/18

(52) U.S. Cl. CPC C07K 16/28; A61P 35/00; C07K 16/18; C07K 2317/24

(57) ABSTRACT

The invention discloses a monoclonal antibody against human GPR48 and application of the same. In the antibody provided by the present invention, the amino acid sequences of LCDR1, LCDR2, and LCDR3 in the light chain variable region are shown in positions 27-32, 50-52, and 89-97 of SEQ ID No.1 sequentially, and the amino acid sequences of HCDR1, HCDR2 and HCDR3 in the heavy chain variable region are shown in positions 26-33, 51-60 and 99-108 of SEQ ID No.2 sequentially. The antibody has strong specificity and sensitivity to GPR48 protein, and can be applied to immunological experimental techniques such as western blotting, immunofluorescence and immunohistochemistry; it can be applied to flow cytometry experimental techniques such as flow cyometry analysis and flow cytometry sorting; it can be applied to the scientific research of signaling pathways such as subcellular localization and protein interaction. The antibody can be used for clinical drug development of human GPR48 is positive tumors such as colorectal cancer.

### RELATED APPLICATIONS

The present application is a U.S. National Phase of International Application Number PCT/CN2022/074456 filed Jan. 28, 2022, and claims priority to Chinese Application Number 202110147974.2 filed Feb. 3, 2021.

### INCORPORATION BY REFERENCE

The sequence listing provided in the file entitled C6351-103_SQL_mod.txt, which is an ASCII text file that was created on Aug. 1, 2023, and which comprises 3,737 bytes, is hereby incorporated by reference in its entirety.

### TECHNICAL FIELD

The invention relates to the field of biotechnology, in particular to a monoclonal antibody against human GPR48 and application thereof.

### BACKGROUND OF THE INVENTION

Colorectal cancer is one of the most common malignant tumors and the third leading cause of death in the world. Targeting tumor therapy is a new method of cancer treatment developed in recent years. It largely avoids the spread of tumor cells and the generation of severe side effects caused by traditional cancer therapies such as surgery, chemotherapy and radiotherapy. Monoclonal antibody therapy against tumor cell surface antigens is a main way of tumor targeting therapy, and has shown good tumor therapeutic effect.

G Protein coupled receptor 48 (GPR48), also known as Leucine Rich Repeat Containing G Protein coupled Receptor 4, GPR48, is a seven-transmembrane protein widely expressed on the surface of eukaryotic cells, a total of 951 amino acids, of which the N-terminal is located outside the cell membrane, and the C-terminal transmembrane 7 times, with a molecular weight of about 104 KD. The gene encoding human GPR48 consists of 106,827 bases and is located in the p14.1 region of chromosome 11. Unlike classical G protein coupled receptors, GPR48 does not mediate downstream signaling through activation of G protein heterotrimers. GPR48 activates the canonical Wnt signaling pathway and participates in the development of various organs. According to the previous research in the applicant's laboratory and other literature reports, it is found that G protein coupled receptor 48 is closely related to the occurrence and development of various malignant tumors such as colorectum cancer, and its main features are: (1) G protein coupled receptor 48 is highly expressed in colorectal cancer, prostate cancer, gastric cancer, lung cancer, breast cancer and other cancer tissues; (2) G protein coupled receptor 48 is co-expressed with human cellular prion protein PrPc in various cancer tissues, such as colorectal cancer, prostate cancer, gastric cancer, lung cancer, breast cancer and other cancer tissues; (3) in animal models, the knockdown of G protein coupled receptor 48 significantly inhibit colorectal tumor growth and liver metastasis; (4) the expression of exogenous G protein coupled receptor 48 significantly promotes tumorigenesis and growth. In summary, G protein coupled receptor 48 can be used as a therapeutic target for various malignant tumors such as colorectal cancer, prostate cancer, gastric cancer, lung cancer, and breast cancer. The previous work of the applicant also show that the cells co-expressing GPR48 and PrPc have the characteristics of tumor stem cells. Therefore, the research on targeted therapy based on monoclonal antibodies against GPR48 is expected to provide a new solution for cancer treatment.

### SUMMARY OF THE INVENTION

The object of the present invention is to provide a monoclonal antibody against human GPR48 and the application of the same.

In a first aspect, the invention claims an antibody.

The antibody claimed in the present invention is a monoclonal antibody against GPR48. The amino acid sequences of LCDR1, LCDR2 and LCDR3 in the light chain variable region of the antibody are shown in positions 27-32, 50-52 and 89-97 of SEQ ID No.1 sequentially. The amino acid sequences of HCDR1, HCDR2 and HCDR3 in the heavy chain variable region of the antibody are shown in positions 26-33, 51-60 and 99-108 of SEQ ID No.2.

Among them, HCDR1, HCDR2 and HCDR3 are the three complementarity determining regions in the heavy chain variable region, and LCDR1, LCDR2 and LHCDR3 are the three complementarity determining regions in the light chain variable region. The sequences of the CDRs are defined according to Kabat.

Within the protection scope of the present invention, the antibody can be in various forms such as a full-length antibody, a Fab fragment, a F(ab′)2 fragment or a single-chain Fv fragment.

Further, the amino acid sequence of the light chain variable region is shown in SEQ ID No.1, or that has an identity of 99% or more, 95% or more, 90% or more, 85% or more, 80% or more or 75% or more to SEQ ID No.1 (inconsistencies can be in the framework region (FR)). The amino acid sequence of the heavy chain variable region is shown in SEQ ID No.2, or has an identity of 99% or more, 95% or more, 90% or more, 85% or more, 80% or more or 75% or more to SEQ ID No.2 (the inconsistency can be in the framework region (FR)).

In a specific embodiment of the present invention, the heavy chain type of the antibody is specifically IgG1; the light chain type of the antibody is specifically λ; and the antibody is specifically a murine antibody.

In the second aspect, the present invention claims a hybridoma cell line.

The hybridoma cell line claimed in the present invention is specifically the mouse hybridoma cell line 1H1, whose registration number in the General Microbiology Center of China Committee for the Collection of Microorganisms is CGMCC No. 20290.

In the third aspect, the present invention claims to protect a monoclonal antibody secreted and produced by the hybridoma cell line 1H1.

The monoclonal antibody is an anti-GPR48 monoclonal antibody (i.e., 2-1H1 antibody in the examples).

In the fourth aspect, the present invention claims to protect an antibody obtained after immunizing an animal (such as a mouse) with a conjugate of the polypeptide shown in SEQ ID No. 5 and a carrier protein (such as KLH) as an immunogen.

The antibody is an anti-GPR48 antibody.

In a specific embodiment of the present invention, the antibody is a monoclonal antibody.

In the fifth aspect, the present invention claims any of the following biological materials:
(A1) A humanized antibody obtained by humanizing the antibody described in the first aspect above, the monoclonal antibody described in the third aspect above, or the antibody described in the fourth aspect above.
(A2) An active fragment of the antibody derived from the antibody described in the first aspect above, the monoclonal antibody described in the third aspect above, the antibody described in the fourth aspect above or the humanized antibody in (A1): a single chain antibody, a Fab region of an antibody, an antigen-binding fragment, etc.
(A3) A nucleic acid molecule encoding the antibody described in the first aspect above, the monoclonal antibody described in the third aspect above, the antibody described in the fourth aspect above, or the humanized antibody in (A1) or the active fragment of the antibody in (A2).
(A4) An expression cassette, a recombinant vector, a recombinant cell or a recombinant bacterium containing the nucleic acid molecules in (A3).
(A5) A kit containing the antibody described in the first aspect above, the monoclonal antibody described in the third aspect above, the antibody described in the fourth aspect above, or the humanized antibody in (A1) or the active fragment of the antibody in (A2), the nucleic acid molecule in (A3), or the expression cassette, the recombinant vector, the recombinant cell or the recombinant bacterium in (A4).

A kit containing antibodies can be used to detect GPR48 protein, specifically, it can be an immunoblotting kit, an immunohistochemistry kit, an immunofluorescence kit, a flow cytometry kit, and the like.
(A6) A polypeptide shown in SEQ ID No. 5.
(A7) A conjugate of the polypeptide shown in (A6) and a carrier protein (such as KLH).

Further, in the nucleic acid molecule in (A3), the nucleotide sequences encoding LCDR1, LCDR2 and LHCDR3 in the light chain variable region of the antibody can be shown in positions 79-96, 148-156, and 265-291 in SEQ ID No. 3 from the 5′ end. In the nucleic acid molecule in (A3), the nucleotide sequences encoding HCDR1, HCDR2 and HCDR3 in the heavy chain variable region of the antibody can be shown in positions 76-99, 151-180 and 295-324 in SEQ ID No. 4 from the 5′ end.

Furthermore, in the nucleic acid molecule of (A3), the nucleotide sequence encoding the light chain variable region of the antibody can be shown in SEQ ID No.3 or has an identity of 99% or more, 95% or more, 90% or more, 85% or more, 80% or more or 75% or more to SEQ ID No.3 (the inconsistency can be in the framework region (FR)). The nucleotide sequence encoding the heavy chain variable region of the antibody is shown in SEQ ID No.4 or has an identity of 99% or more, 95% or more, 90% or more, 85% or more, or 80% or more, or 75% or more to SEQ ID No.4 (the inconsistency can be in the framework region (FR)).

In the sixth aspect, the present invention claims a pharmaceutical composition.

The pharmaceutical composition claimed in the present invention comprises the antibody described in the first aspect above, the monoclonal antibody described in the third aspect above, the antibody described in the fourth aspect above, the humanized antibody described in the fifth aspect (A1) above or the active fragment of the antibody described in the fifth aspect (A2).

Further, the pharmaceutical composition may also contain an anti-tumor drug.

In a specific embodiment of the present invention, the anti-tumor drug is specifically the cellular prion protein PrPc monoclonal antibody Ab-5 (a monoclonal antibody against cellular prion protein, obtained from a hybridoma cell line with a deposit number of CGMCC No. 4972, Chinese Invention Patent No. 201110284158.2, Invention Title Application of anti-cellular prion protein monoclonal antibody in preparing an anti-tumor drug).

In a specific embodiment of the present invention, the mass ratio of the monoclonal antibody (i.e., 2-1H1 antibody) to the cellular prion protein PrPc monoclonal antibody Ab-5 is 1:1.

In the seventh aspect, the present invention claims to protect any one of the following applications:
(B1) Application of the nucleic acid molecule described in (A3) of the fifth aspect above, the expression cassette, the recombinant vector, the recombinant cell or the recombinant bacterium described in (A6) of the fifth aspect or the conjugate described in (A7) of the fifth aspect in preparing the antibody described in the first aspect above, the monoclonal antibody described in the third aspect above, the antibody described in the fourth aspect above or the humanized antibody described in (A1) of the fifth aspect or the active fragment of the antibody described in (A2) of the fifth aspect or the kit in (A5) of the fifth aspect or the pharmaceutical composition described in the sixth aspect above.

Wherein, the polypeptide in (A6) or the conjugate in (A7) is used as an immunogen to immunize animals (such as mice) to prepare an antibody.
(B2) Application of the antibody described in the first aspect above, the hybridoma cell line 1H1 described in the second aspect above, the monoclonal antibody described in the third aspect above, the antibody described in the fourth aspect above, the humanized antibody described in the fifth aspect (A1) above, the active fragment of the antibody in the fifth aspect (A2) in the preparation of the pharmaceutical composition described in the sixth aspect above.
(B3) Application of the antibody described in the first aspect above, the hybridoma cell line 1H1 described in the second aspect above, the monoclonal antibody described in the third aspect above, the antibody described in the fourth aspect above, the biological material described in the fifth aspect above, or the pharmaceutical composition described in the sixth aspect in the preparation of an anti-tumor product.
(B4) Application of the antibody described in the first aspect above, the hybridoma cell line 1H1 described in the second aspect above, the monoclonal antibody described in the third aspect above, the antibody described in the fourth aspect above, the biological material described in the fifth aspect above, or the pharmaceutical composition described in the sixth aspect in the preparation of a product for inhibiting tumor growth.
(B5) Application of the antibody described in the first aspect above, the hybridoma cell line 1H1 described in the second aspect above, the monoclonal antibody described in the third aspect above, the antibody described in the fourth aspect above, the biological material described in the fifth aspect above, or the pharmaceutical composition described in the sixth aspect in the preparation of a product for inhibiting tumor metastasis.
(B6) Application of the antibody described in the first aspect above, the hybridoma cell line 1H1 described in the second aspect above, the monoclonal antibody described in the third aspect above, the antibody described in the fourth aspect above, the biological material described in the fifth aspect above, or the pharmaceutical composition described in the sixth aspect in the preparation of a product for inhibiting the growth of tumor organoids.
(B7) Application of the antibody described in the first aspect above, the hybridoma cell line 1H1 described in the second aspect above, the monoclonal antibody described in the third aspect above, the antibody described in the fourth aspect above, the biological material described in the fifth aspect above, or the pharmaceutical composition described in the sixth aspect in the preparation of a product for inhibiting the formation of tumor cell clones.
(B8) Application of the antibody described in the first aspect above, the hybridoma cell line 1H1 described in the second aspect above, the monoclonal antibody described in the third aspect above, the antibody described in the fourth aspect above, the biological material described in the fifth aspect above, or the pharmaceutical composition described in the sixth aspect in the preparation of a product for inhibiting GPR48.
(B9) Application of the antibody described in the first aspect above, the hybridoma cell line 1H1 described in the second aspect above, the monoclonal antibody described in the third aspect above, the antibody described in the fourth aspect above, the biological material described in the fifth aspect above, or the pharmaceutical composition described in the sixth aspect in the preparation of a product for detecting GPR48.
(B10) Application of the antibody described in the first aspect above, the hybridoma cell line 1H1 described in the second aspect above, the monoclonal antibody described in the third aspect above, the antibody described in the fourth aspect above, the biological material described in the fifth aspect above, or the pharmaceutical composition described in the sixth aspect in the preparation of a product for inhibiting the viability of GPR48 positive cells.

Wherein, the GPR48 positive cell may be a tumor cell expressing GPR48 (such as a GPR48 positive colorectal cancer cell).

In a specific embodiment of the present invention, the GPR48 positive cells are specifically GPR48 positive colorectal cancer cells SW480, HCT116 and P6C.
(B11) Application of the antibody described in the first aspect above, the hybridoma cell line 1H1 described in the second aspect above, the monoclonal antibody described in the third aspect above, the antibody described in the fourth aspect above, the biological material described in the fifth aspect above, or the pharmaceutical composition described in the sixth aspect in the preparation of a product for inhibiting WNT signaling pathway.
(B12) Application of the antibody described in the first aspect above, the hybridoma cell line 1H1 described in the second aspect above, the monoclonal antibody described in the third aspect above, the antibody described in the fourth aspect above, the biological material described in the fifth aspect above, or the pharmaceutical composition described in the sixth aspect in the preparation of a product for inhibiting the expression of Ki67 protein, LRP6 protein and/or β-catenin protein.
(B13) Application of the antibody described in the first aspect above, the monoclonal antibody described in the third aspect above, the antibody described in the fourth aspect above or the humanized antibody described in (A1) of the first aspect above or the active fragments of antibodies described in (A2) of the first aspect above in the preparation of synergistic auxiliary preparations of an anti-tumor drug.

Further, the anti-tumor drug can be cellular prion protein PrPc monoclonal antibody Ab-5 (a monoclonal antibody against cellular prion protein, produced by a hybridoma cell line with a deposit number of CGMCC 4972, Chinese Invention Patent No. 2011102841582, Invention Title: Application of anti-cellular prion monoclonal antibody in the preparation of an anti-tumor drug).

In the above aspects, the tumor can specifically be a GPR48 positive solid tumor; the tumor organoid is a GPR48 positive solid tumor organoid; and the tumor cells are GPR48 positive tumor cells.

Further, the solid tumor may specifically be colorectal cancer, breast cancer, prostate cancer, pancreatic cancer, gastric cancer, and the like.

In a specific embodiment of the present invention, the tumor cells are specifically colorectal cancer cells SW480, HCT116, HT29 or P6C. The tumor organoid is specifically a colorectal cancer organoid.

The tumor organoid is preferably a solid tumor organoid cultured in 3D in vitro.

### STATEMENT ON DEPOSITION

Reference biological material (strain): 1H1

Scientific Description: Mouse Hybridoma

Deposition institution: General Microbiology Center of China Committee for the Collection of Microorganisms

Depository institution abbreviation: CGMCC

Address: No. 3, Yard No. 1, Beichen West Road, Chaoyang District, Beijing

Deposition date: Sep. 3, 2020

Deposit number of the collection center: CGMCC No. 20290

### BRIEF DESCRIPTION OF THE DRAWINGS

FIGS. 1A and 1B are graphs showing the specific binding of 2-1H1 antibody to GPR48. FIG. 1A. Binding of 2-1H1 antibody (left) and commercial GPR48 monoclonal antibody (R&D Company, middle) to colon cancer cell line P6C endogenous GPR48 by flow cytometry detection, IgG is used as a negative control (right); FIG. 1B. Binding of anti-2-1H1 antibody to wild-type P6C (left) and P6C-GPR48 exogenously expressing GPR48 (middle) by flow cytometry detection.

FIGS. 2A and 2B show the application of 2-1H1 antibody in western blot and immunohistochemistry. FIG. 2A. Binding of 2-1H1 antibody to GPR48 in four colorectal cancer cell lines by immunoblot detection, wherein LGR4 in FIG. 2B is GPR48; FIG. 2B. Binding of antibody to GPR48 in colon cancer specimens by immunohistochemistry.

FIGS. 3A-3C are immunofluorescence graph showing that 2-1H1 antibody recognizes the colon cancer organoid cell membrane protein GRP48. FIG. 3A. GRP48 is localized on the plasma membrane of colon cancer cells; FIG. 3B. DAPI-labeled nuclear staining; FIG. 3C. Membrane protein GPR48 overlaps with the image of the nucleus.

FIGS. 4A-4C show the expression of GPR48 in different tissues detected by flow cytometry. FIG. 4A. Normal colon; FIG. 4B. Colon cancer; FIG. 4C. Colon cancer organoids.

FIGS. 5A-5C show the GPR48 expression in different tissues by immunofluorescence. The first row shows FIG. 5A. GPR48 in normal colon; FIG. 5B. GPR48 in colon cancer; FIG. 5C. GPR48 in colon cancer organoids; the second row shows the overlap of GPR48 and corresponding nuclei.

FIGS. 6A-6C show the effect of 2-1H1 antibody on the viability of three colorectal cancer cells. FIG. 6A. Treatment time 24 h; FIG. 6B. Treatment time 48 h; FIG. 6C. Treatment time 72 h. LGR4 in FIG. 6 is GPR48.

FIG. 7A and 7B show the inhibitory effect of 2-1H1 antibody on the growth of tumor organoids. FIG. 7A. Bright field image of tumor organoids (the first row from top to bottom shows 1#-5# tumor organoids co-cultured with 2-1H1 antibody for 14 days, the final antibody concentration is 10 μg/ml, and the second row shows 1#-5# tumor organoids co-cultured with mouse IgG for 14 days, and the IgG concentration is 10 μg/ml); FIG. 7B. Statistical graph of the number of tumor organoids.

FIGS. 8A and 8B show the in vivo therapeutic effect of 2-1H1 antibody on tumors in mice. FIG. 8A. In situ tumor image, the first row shows the IgG control group; the second row shows 2-1H1 monoclonal antibody experimental group; FIG. 8B. The statistics of the in situ tumor volume.

FIGS. 9A-9C show that the combination treatment of 2-1H1 and PrPc monoclonal antibody Ab-5 can better inhibit the growth of in situ tumor and the ability of tumor metastasis. FIG. 9A. Fluorescence of abdominal tumors of mice treated alone or in combination; FIG. 9B. Tumor volume of mice treated alone or combined (the first row from top to bottom shows the mouse colorectal tumors injected intraperitoneally with IgG, the first row shows the mouse colorectal tumors injected intraperitoneally with 2-1H1 monoclonal antibody, the third row shows the mouse colorectal tumors injected intraperitoneally with PrPc monoclonal antibody Ab-5, and the fourth row shows the mouse colorectal tumors injected intraperitoneally with 2-1H1 and Ab-5); FIG. 9C. H&E staining of livers of mice treated alone or in combination.

FIGS. 10A-10C show that 2-1H1 inhibits the expression of Ki67 in tumor organoids (FIG. 10A), the expression levels of LRP6 and β-catenin (FIG. 10B) after 12 and 24 hours of 2-1H1 treatment of tumor organoids, IgG is used as a negative control, β-actin is used as a sample volume reference. FIG. 10C shows 2-1H1 treatment of HCT116 cell line inhibits the expression of LRP6 and β-catenin.

FIGS. 11A and 11B show the degradation of GPR48 caused by 2-1H1 monoclonal antibody. FIG. 11A shows the expression level of GPR48 detected by western blot, after different time of 2-1H1 monoclonal antibody treatment of tumor cells. FIG. 11B shows 2-1H1 promotes the degradation of GPR48 on the cell membrane as detected by immunofluorescence, after the incubation of 2-1H1 monoclonal antibody with tumor cells.

### BEST MODE OF IMPLEMENTING THE INVENTION

The following examples facilitate a better understanding of the present invention, but do not limit the present invention. The experimental methods in the following examples are conventional methods unless otherwise specified. The test materials used in the following examples, unless otherwise specified, are purchased from conventional biochemical reagent stores. Quantitative experiments in the following examples are all set up to repeat the experiments three times, and the results are averaged.

Cell lines and main reagents used in the following examples:

The cell lines used in the present invention include: P6C (this cell line is isolated from the tumor tissue of colon cancer patients by our laboratory. Chinese invention patent No. 201210033124.0, invention title: a human colorectal cancer tumor cell line and its preparation method and application, the deposition number is CGMCC No. 5558), SW480 (ATCC® CCL-228™), HCT116 (ATCC® CCL-247™), HT-29 (ATCC® HTB-38™) are purchased for this laboratory and have been used, cultivated and preserved for a long time.

Main reagent used in the present invention and source thereof are as follows:

Actin monoclonal antibody (A-5441), a-tubulin monoclonal antibody, Triton-X 100, PAGE gel preparation reagents, and Pipes are all from Sigma.

Commercial GPR48 monoclonal antibody is a product of R&D Systems, clone #852229, product number MAB7750. The commercial GPR48 polyclonal antibody is a product of ThermoFisher Company, catalog number PA527177. The FITC-labeled anti-mouse fluorescent secondary antibody is a product of ThermoFisher Company, and the horseradish peroxidase-coupled secondary antibody is a product of KPL Company.

EMEM cell culture medium, OPTI-MEM, cellulose acetate membrane for Western blotting, Hepes, 100×GlutaMax, 100×N2, 50×B27 are all from Gibco.

Coomassie Blue G250, R250, NP-40, Tween 20, and DMSO are from Merck.

Chromogenic substrate AB solution is from Pierce.

The PfuUltra High-Fidelity DNA Polymerases II and DNA ligase used in constructing the plasmids are from Takara Company.

Restriction enzymes are from NEB Company.

Lipofectemin 2000 is from Invitrogen.

FBS (Fetal bovine serum) is from Hyclone.

All other reagents are of domestic analytical grade unless otherwise specified.

### EXAMPLE 1. SYNTHESIS OF GPR48 ANTIGEN POLYPEPTIDE TARGET

1. Design and synthesize the polypeptide QLPEDAFKNFPFLEE (SEQ ID No.5) according to the amino acid sequence of human GPR48.
2. 1 mg of polypeptide QLPEDAFKNFPFLEE is coupled with 1 mg of carrier protein KLH to be an immunogen.

The specific coupling method is:
Dissolve 1 mg of KLH in 200 μl of PBS;
Dissolve 0.2 mg of Sulfo-SMCC in 40 μl of PBS;
Then slowly drop the dissolved SMCC solution into the KLH solution, mix gently while adding, and react at room temperature for 1 hour;
Add the solution to a dialysis bag, dialyze with PBS to remove excess SMCC, and dialyze overnight;
On the second day, after changing the dialysate 1-2 times, slowly drop 1 mg of polypeptide dissolved in 200 μl of PBS into the semi-conjugate, mix well while adding, and react at room temperature for 4 hours;
After the reaction is completed, the coupling product is stored in separate packages.

Identification of coupling results: add 100 μl of Ellman reagent stock solution to a 96-well plate, then add 10 μl of coupled peptide solution, and measure the UV absorbance at λ=412 nm with a spectrophotometer. An OD value<0.03 indicates the polypeptide and KLH protein is coupled successfully.
3. 0.5 mg of polypeptide QLPEDAFKNFPFLEE is coupled with 0.5 mg of carrier protein BSA to be a detection source.

The specific coupling method is:
Dissolve 0.5 mg of BSA in 100 μl of PBS;
Dissolve 0.1 mg of sulf-SMCC in 20 μl of PBS;
Then slowly drop the dissolved SMCC solution into the BSA solution, mix gently while adding, and react at room temperature for 1 hour;
Add the solution to a dialysis bag, dialyze with PBS to remove excess SMCC, and dialyze overnight;
On the second day, after replacing 1-2 dialysate, slowly drop 1 mg of polypeptide dissolved in 200 μl of PBS into the semi-conjugate, mix well while adding, and react at room temperature for 4 hours;
After the reaction is completed, store in aliquots.

Identification of coupling results: add 100 μl of Ellman reagent stock solution to a 96-well plate, then add 10 μl of coupled peptide solution, and measure the UV absorbance at λ=412 nm with a spectrophotometer. OD value<0.03 indicates that the peptide and BSA protein is coupled successfully.

### EXAMPLE 2. PREPARATION OF A MOUSE ANTI-HUMAN GPR48 ANTIBODY

### 1. Immunization of Animals

The KLH-coupled QLPEDAFKNFPFLEE peptide (210 μg of the polypeptide shown in SEQ ID No.5) is dissolved in 600 μl of PBS buffer, mixed with 600 μl of a complete Freund's adjuvant (Sigma, product number F5881), and placed in the syringe, pushed back and forth for 5-10 minutes to obtain a stable emulsion, that is, an immunogen. Subcutaneously immunize 3 SPF-grade Balb/c female mice (hereinafter referred to as polypeptide-immunized mice), numbers: 1, 2, and 3; the amount of immunization is 70 μg protein/mouse (based on the mass of the polypeptide shown in SEQ ID No.5). The mouse tail blood is drawn on the 14^{th} day after immunization, and the antibody titer of the tail blood is evaluated by ELISA method.

### 2. Potency Detection

Steps: Use the peptide in Example 1, peptide-coupled BSA (the coupling agent is SMCC), and the coupling agent+BSA to coat the microtiter plate (1 μg/mL), add 100 μL to each well, and react overnight at 4° C.; wash the plate 3 times with PBS solution, block with 5% milk-PBS at room temperature for 1 hour; then wash the plate once with PBS solution, add mouse tail blood serially diluted with 5% milk-PBS solution, and react at room temperature for 1 hour; after washing the plate with PBS solution for 3 times and patting dry, add 1:2000 diluted HRP-labeled goat anti-mouse IgG (Fc) secondary antibody and react at room temperature for 1 hour; after washing the plate with PBS solution for 5 times, pat dry, add an equal volume of TMB Chromogenic Reagent (Biyuntian, Cat. No. P0209), and react at room temperature for 20 minutes in the dark; then add 50 μL of stop solution (Biyuntian, Cat. No. P0215), and read the OD450 value on a microplate reader after mixing well.

The results show that the tail blood of the three mice in the peptide immunization group all recognize the peptide-conjugated BSA (polypeptide-BSA), and the titers all reach 1:50000, which is higher than that of the unconjugated polypeptide (1:5000), indicating that the peptide-conjugated BSA enhances the coating effect. At the same time, the signal for tail blood of the peptide-immunized mice recognizing the coupling agent-BSA is weak, indicating that most of the tail blood of the mice are antibodies that specifically recognize the polypeptide.

### 3. Cell Fusion Experiments

According to the above ELISA results, the No. 1 mouse with the highest titer is selected for cell fusion. Mouse splenocytes and SP2/0 cells are fused by PEG method.

### 4. Pick Clones

After fusion, 564 single clones are picked and cultured in six 96-well cell culture plates.

### 5. The First Screening of Monoclonal Cells

The detection source (polypeptide QLPEDAFKNFPFLEE coupled with BSA) in Example 1 is used to coat the plates, and the culture supernatants of 564 cells are evaluated by the above-mentioned ELISA method. The cell culture supernatant is screened for monoclonal cell lines that could recognize the polypeptide-BSA. By ELISA method, 8 positive monoclonal cells with OD450 value higher than 0.8 are screened out.

### 6. The Second Screening of Monoclonal Cells

The 8 positive monoclonal cells are expanded into 48-well plates, and after 2 days of culture, the ELISA test is repeated with the detection source, and the second screening is performed to obtain 5 positive hybridoma cell lines with an OD450 value higher than 1.

### 7. The Third Screening of Monoclonal Cells

The 5 positive clones from the second screening are further expanded into a 12-well plate, the cell supernatant is collected, centrifuged at 12000 rpm for 10 minutes, and the supernatant is taken. Incubate with P6C cells at 4° C. for 30 minutes, add goat anti-mouse IgG-FITC, and incubate at 4° C. for 30 minutes. The proportion and fluorescence intensity of FITC-positive cells are detected by flow cytometry, and two monoclonal cells, 1H1 and 6E11, secreting antibodies specifically recognize the endogenous GPR48 protein on the surface of P6C cells are obtained. Among them, the average fluorescence intensity of the positive signal detected by 1H1 is 50.1, which is significantly higher than the average fluorescence intensity detected by 6E11, which is 25.18.

### 8. Identification of Monoclonal Cell Subtypes

The culture supernatants of the two positive cell lines obtained from the third screening are subtype identified with a kit, and finally the antibody subtype secreted by 1H1 cells is found to be IgG1, and the light chain type is λ.

### 9. Freezing

Preparation of cryopreservation solution, FBS:IMDM:DMSO (volume ratio)=8:1:1;

Three tubes are frozen for each strain, and another three tubes are frozen again after 3 days. The freezing method is the freezing box freezing method, after overnight at −80° C., they are stored in a liquid nitrogen cabinet.

### 10. Sequencing

RNA extraction, reverse transcription, and PCR amplification and sequencing of antibody variable regions are carried out on the cell lines with strong antibody secretion ability, and we obtain the cDNA sequence of the variable region of the monoclonal antibody anti-GPR48 m2-1H1 (referred to as 2-1H1) secreted by the mouse hybridoma cell line 1H1 as follows:
GPR48 2-1H1 VH (heavy chain variable region): SEQ ID No. 4.
GPR48 2-1H1 VL (light chain variable region): SEQ ID No. 3.

The corresponding amino acid sequences are as follows:
GPR48 2-1H1 VH (heavy chain variable region): SEQ ID No. 2.
GPR48 Ab-L5 VL (light chain variable region): SEQ ID No. 1.

### EXAMPLE 3. PREPARATION OF 2-1H1 MONOCLONAL ANTIBODY

The mouse hybridoma cell line 1H1 in Example 2 is deposited in the General Microbiology Center of China Committee for the Collection of Microorganisms, with the date of deposit on Sep. 3, 2020, and the deposit number is CGMCC No. 20290.

The mouse hybridoma cell line 1H1 is used to prepare the mouse anti-GPR48 monoclonal antibody 2-1H1, and the specific operation steps are as follows:

### 1. Cell Recovery

Quickly take out a frozen tube from the −80° C. refrigerator or liquid nitrogen tank; quickly put it into a 37° C. water bath and stir quickly to make the cryoprotectant melt into a liquid within 2 minutes. Add 3 mL of IMDM medium containing 15% FBS serum to a 15 mL centrifuge tube, draw the cryoprotectant into the centrifuge tube, centrifuge at 1500 rpm for 5 minutes. Discard the supernatant, suspend the cells with complete medium, and culture in a 6-well plate (3 mL) or bottle (5 mL).

### 2. Preparation of Ascites

The cells in the logarithmic growth phase are washed with serum-free medium and suspended; the count is about 5×10⁵, 1 mL. The suspended cells are injected intraperitoneally into mice. Ascites is collected after 7-10 days. Collect about 3 mL for the first time, repeat the collection every 2 or 3 days, and finally collect 8 mL/mice. Each ascites taken out is centrifuged at 4000 rpm for 10 minutes; the middle is ascites. Carefully suck out the ascites and collect it in a centrifuge tube for the next step of Protein G purification.

### 3. Purification of Monoclonal Antibodies

Take 1 mL of the column material coupled with protein G and add it to an empty column, wash it with PBS solution, dilute 2 mL of ascites with 8 mL of PBS and load it on the column, then put the flow-through solution on the column again; then glycine eluent (0.1 M glycine-HCl) at pH 2.7 is used for elution, and 100 μL neutralizing solution (1.0M Tris-HCl, pH 9.0) is added to each 1 mL eluent collection tube in advance, and 5 tubes are collected in total. Then glycine eluent (0.1 M glycine-HCl) at pH 1.9 is used for elution, collect one tube per 1 mL of eluent (300 μL neutralizing solution, 1.0M Tris-HCl, pH 9.0, is added in advance), a total of 3 tubes are collected; then read the OD280 of each tube of eluate separately, mix the eluent with OD280 greater than 0.5, re-measure the OD280 of the mixture after mixing, and calculate the antibody concentration according to the coefficient of 1.4. Antibody concentration=OD280/1.4.

### EXAMPLE 4. 2-121 MONOCLONAL ANTIBODY SPECIFICALLY RECOGNIZES GPR48

### 1. Determination of Monoclonal Antibody Titer

2-1H1 monoclonal antibody prepared by the method of the present invention is evaluated by the above-mentioned ELSIA method, and it is found that the antibody recognize the polypeptide QLPEDAFKNFPFLEE coupled to BSA, and the sensitivity is greater than 0.0005 μg/mL (5 times greater than that of the negative control, 0.372/0.067=5.55), and no cross-reaction with coupling agent-BSA (Table 1).

TABLE 1
OD₄₅₀, dilution unit μg/mL

| | | | | | | | Negative |
|---|---|---|---|---|---|---|---|
| Coating | Dilution | 1 | 0.5 | 0.05 | 0.005 | 0.0005 | Control |
| Peptide | 1H1 | 0.051 | 0.052 | 0.05 | 0.053 | 0.056 | 0.066 |
| Peptide-BSA | | 999 | 3.27 | 3.335 | 2.397 | 0.372 | 0.067 |
| SMCC-BSA | | 0.055 | 0.063 | 0.056 | 0.057 | 0.049 | 0.069 |

2. Dilute 1 mg/ml of 2-1H1 monoclonal antibody prepared by the method of the present invention and commercial mouse anti-GPR48 monoclonal antibody (R&D Company), rabbit anti-GPR48 polyclonal antibody (ThermoFisher Company), respectively at 1:100, 1:300, 1:500, 1:1000, 1:2000, 1:4000, 1:10000 and incubate overnight with colon cancer cell P6C lysate electrophoresis transfer membrane, and then incubate with horseradish peroxidase-labeled secondary antibody for 4 hours, incubate with ECL luminescent substrate for exposure and development, and detect the sensitivity of the antibody. The result shows that the titer of 2-1H1 monoclonal antibody prepared by the method of the present invention is 13.3 times that of the commercial GPR48 monoclonal antibody, and 4 times that of the commercial GPR48 polyclonal antibody (the titer of 2-1H1 monoclonal antibody prepared by the method of the present invention is 1:4000, the titer of commercial monoclonal antibody is 1:300, and the titer of commercial polyclonal antibody is 1:1000. That is, 4000/300=13.3, 4000/1000=4).

### 2. Determination of the Specificity and Sensitivity of the Antibody

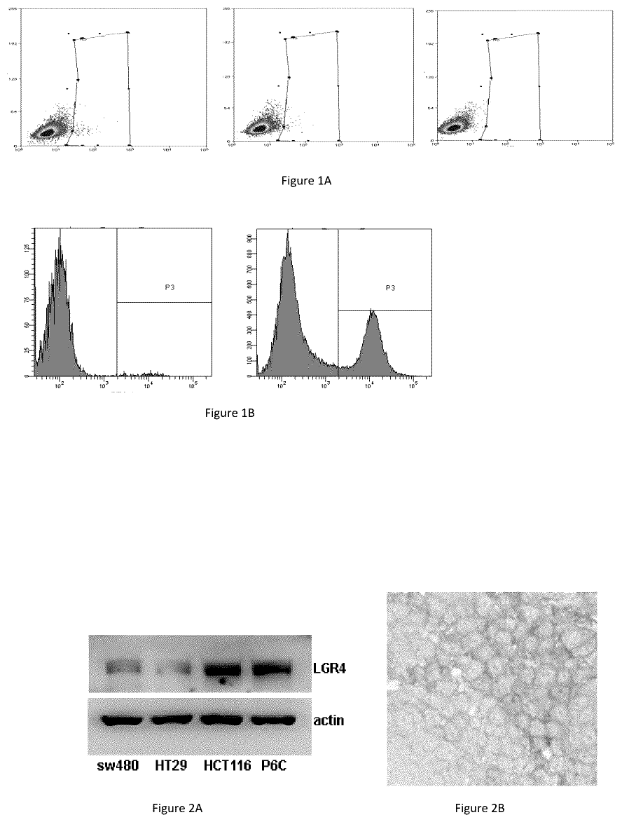

Colon cancer cell line P6C is cultured overnight, digested with trypsin substitute TrypLE Express at 37° C. for 5 minutes, and serum is added to stop the reaction. Washing with PBS 3 times, and after diluting 2-1H1 monoclonal antibody prepared by the method of the present invention and the commercial mouse anti-GPR48 protein monoclonal antibody (R&D Company) respectively at 1:100, 1:200, 1:500, 1:1000, and 1:2000, incubate with colon cancer cell line P6C for 30 minutes, and then incubate with FITC fluorescently labeled goat anti-mouse IgG for 30 minutes. Fluorescence intensity is detected by flow cytometry, and the sensitivity of the antibody is detected. The sensitivity of 2-1H1 monoclonal antibody prepared by the method of the present invention is 5 times that of the commercial GPR48 monoclonal antibody (the dilution factor of 2-1H1 monoclonal antibody prepared by the method of the present invention is 500, and the dilution factor of the commercial antibody is 100. That is, 500/100=5) (A in FIG. 1).

The GPR48 gene (GeneID: 55366) is inserted into the multiple cloning site of HindIII and EcoI in pcDNA4-TO-myc-his-B vector (Ziheng Chen et al. Mitochondrial E3 ligase MARCH5 regulates FUNDC1 to fine-tune hypoxic mitophagy. EMBO Rep. 2017 March; 18(3): 495-509.), and the correct recombinant vector verified by sequencing is used to transfect the colon cancer cell line P6C with lipo2000 transfection reagent to construct the colon cancer cell line P6C-GPR48 expressing exogenous GPR48. The trypsin substitute TripLE-Express is digested into a single cell suspension, and the 1 mg/ml 2-1H1 monoclonal antibody prepared by the method of the present invention is diluted with a ratio of 1:500, and is respectively incubated with wild-type P6C and P6C-GPR48 cells in PBS phosphate buffer for 30 minutes at 4° C., then incubate with FITC fluorescently labeled goat anti-mouse IgG for 30 minutes. The expression of GPR48 is detected by flow cytometry. As shown in B in FIG. 1, 2-1H1 monoclonal antibody detects an increase in the positive ratio in P6C-GPR48 cells (compared with P6C), indicating that the antibody specifically recognizes GPR48, and also shows that 2-1H1 monoclonal antibody prepared by the method of the present invention specifically recognizes exogenous GPR48.

### EXAMPLE 5. APPLICATION VERIFICATION OF 2-1H1 MONOCLONAL ANTIBODY-WESTERN BLOTTING

### 1. Experimental Steps

Four colorectal cancer cell lines (SW480, HT29, HCT116 and P6C) are lysed, and 30 μg of the whole protein sample in the supernatant of the lysate is loaded on a 12% SDS-PAGE gel; The gel is soaked in 1×transfer solution for 10 minutes after electrophoresis; PVDF membrane is treated with methanol, and transferred to the membrane at 350 mA for 70 minutes; the membrane is blocked in TBST blocking solution containing 5% skimmed milk powder at 37° C. for 1 hour; 2-1H1 monoclonal antibody (1:2000 dilution) is incubated at 37° C. for 1 hour or overnight at 4° C.; corresponding goat anti-mouse secondary antibody (Zhongshan Jinqiao, diluted 1:2500) is incubated at room temperature for 1 hour; after incubation with luminescence reagent (Pierce), X-ray film is exposed.

### 2. Experimental Results

As shown in A of FIG. 2, all four colorectal cancer cell lines expressed GPR48, in which HT29 had a low expression level, SW480 had an intermediate expression level, and HCT116 and P6C expressed higher levels of GPR48.

### EXAMPLE 6. APPLICATION VERIFICATION OF 2-1H1 MONOCLONAL ANTIBODY-IMMUNOHISTOCHEMISTRY (IHC)

### 1. Source of Materials

The colorectal cancer tissue samples used in specific examples of the present invention are purchased from Xi'an Alina Biotechnology Co., Ltd., including colorectal cancer and adjacent normal tissue samples. The samples are collected from surgically resected human colorectal cancer and adjacent tissues, washed with normal saline, fixed in 10% formalin, and preserved in paraffin-embedded sections. All specimens are pathologically confirmed.

### 2. Experimental Steps

Slices are baked, dewaxed in xylene, and hydrated with graded ethanol. Incubate with 0.3% H₂O₂ methanol solution at room temperature for 10 minutes, then wash with 0.01M PBS (phosphate buffered saline) 3 times for 5 minutes; block with 5% skimmed milk powder at 37° C. for 1 hour; incubate with 1 mg/ml anti-GPR48 monoclonal antibody 2-1H1 (diluted in PBS at 1:2000) overnight at 4° C. in a wet box; drop general secondary antibody (Zhongshan Jinqiao, PV-6002) and incubate at room temperature for 30 minutes; color develop with DAB-H₂O₂ (Zhongshan Jinqiao, ZLI-9031), control under the microscope for 1-5 minutes, rinse with tap water, stop color development; counterstain with hematoxylin (Zhongshan Jinqiao, ZLI-9040) for 45 seconds, then differentiate with 1% hydrochloric acid alcohol; rinse with tap water to turn blue; dehydrate with 95% and 100% ethanol for 5 minutes each, transparent in xylene, and seal with neutral gum.

### 3. Experimental Results

As shown in B of FIG. 2, 2-1H1 can be used for immunohistochemistry to identify GPR48 positive cells in paraffin sections of colorectal cancer, and GPR48 is localized in the plasma membrane of the cells.

### EXAMPLE 7. APPLICATION VERIFICATION OF 2-1H1 MONOCLONAL ANTIBODY-DETECTION OF SUBCELLULAR LOCALIZATION OF PROTEIN

### 1. Source of Materials

The colorectal cancer organoids used are obtained from colorectal cancer samples from Beijing Cancer Hospital (all specimens are confirmed by pathology), washed with normal saline, cut into pieces, digested with collagenase and dispase at 37° C. for 2 hours, let stand for 1 minute, and took natural precipitate, washed 3 times with PBS, mix with 100 μl Matrigel (Corning, Cat. No. 356234) on ice, inoculate into 24-well plates, solidify at 37° C. for 15 minutes, and cover with 800 μl colon cancer organoid culture medium (Advanced DMEM/F12+GlutaMax+HEPES−N2+B27), each replace with fresh medium every 2 days.

### 2. Experimental Steps

For staining, organoids (colorectal cancer organoids) are pipetted out of Matrigel, added trypsin substitute TrypLE Express, digested at 37° C. for 5 minutes, centrifuged, and colon cancer organoids are seeded on coverslips. After overnight culture in colon cancer organoid culture medium, transfer cells and coverslips to ice, incubate with 4° C. pre-cooled 2-1H1 monoclonal antibody (diluted 1:1000) on ice for 2 hours, wash with PBS at 4° C. 3 times. Fix with methanol at −20° C. for 20 minutes, wash with PBS three times, add FITC-labeled goat anti-mouse secondary antibody (1:200 dilution) and incubate for 1 hour in a dark box; cells are stained with 0.25 μg/μL 4′,6-diamidino-2-phenylindole (DAPI) solution for 4 minutes; the coverslip is taken out and covered on a slide dripped with 10 μL of anti-fluorescence attenuation mounting medium, and observed under a laser scanning confocal microscope.

### 3. Experimental Results

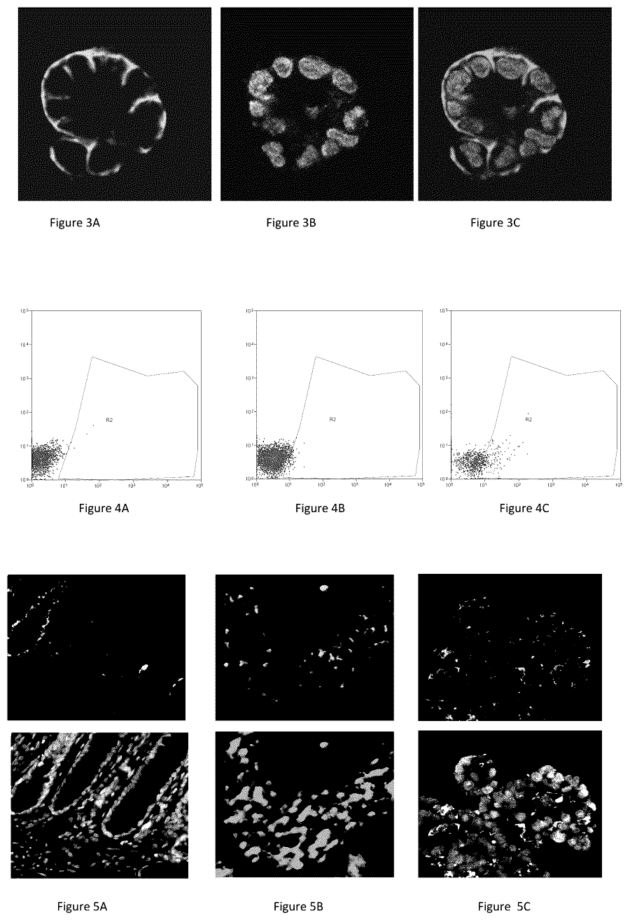

As shown in FIG. 3, 2-1H1 monoclonal antibody recognizes the GPR48 protein localized on the cell membrane.

### EXAMPLE 8. APPLICATION VERIFICATION OF 2-1H1 MONOCLONAL ANTIBODY-FLOW CYTOMETRY (FCM)

### 1. Experimental Steps

The surgically resected fresh colorectal cancer tissue and its corresponding adjacent tissue (sourced from Beijing Cancer Hospital, specimens confirmed by pathology) are washed with normal saline, cut into pieces, digested with collagenase and dispase at 37° C. for 2 hours, and allowed to stand for 1 minute, take the natural precipitation. Add trypsin substitute TrypLE Express and digest at 37° C. for 30 minutes. Filter through a 400-mesh cell sieve and wash twice with phosphate buffered saline. Add 2-1H1 monoclonal antibody 1:500, incubate at 4° C. for 30 minutes, add fluorescently labeled goat anti-mouse, incubate at 4° C. for 30 minutes, and detect the fluorescent signal by flow cytometry.

Colorectal cancer organoids (same as step 1 in Example 7) are separated and digested with TrypLE Express, filtered with a 400-mesh cell sieve, and washed twice with phosphate buffer. Add 2-1H1 monoclonal antibody 1:500, incubate at 4° C. for 30 minutes, add FITC fluorescently labeled goat anti-mouse IgG, incubate at 4° C. for 30 minutes, filter into a single cell suspension with a 400-mesh cell sieve, and detect by flow cytometry fluorescence intensity and proportion of positive cells.

### 2. Experimental results

As shown in FIG. 4, 2-1H1 monoclonal antibody can be used to identify GPR48 in normal colon, primary colon cancer, and colon cancer organoids by flow cytometry.

### EXAMPLE 9. APPLICATION VERIFICATION OF 2-1H1 MONOCLONAL ANTIBODY-IMMUNOFLUORESCENCE (IF)

### 1. Experimental Steps

Colorectal cancer tissues and adjacent tissues are obtained from Xi'an Alina Biotechnology Co., Ltd., and colon cancer organoids (same as step 1 in Example 7) are fixed in formalin, embedded in paraffin, and sectioned at a thickness of 5 μm. Slices are baked, dewaxed in xylene, and hydrated with graded ethanol. Then wash with 0.01M PBS (phosphate buffer saline) 3 times×5 minutes; blocked with 5% skimmed milk powder at 37° C. for 2 hours; incubated with 2-1H1 monoclonal antibody (1:1000 dilution) overnight at 4° C. in a wet box; drop add fluorescently labeled goat anti-mouse secondary antibody (Invitrogen), and incubate at room temperature for 30 minutes. 0.25 μg/μL 4′,6-diamidino-2-phenylindole (DAPI) solution is used to stain the cells for 4 minutes; the coverslip is taken out and covered on a glass slide dripped with 10 μL anti-fluorescence fading mounting medium, laser fluorescent signals are observed under a scanning confocal microscope.

### 2. Experimental Results

As shown in FIG. 5, 2-1H1 monoclonal antibody can be used for immunofluorescence recognition of GPR48 protein in normal colon, colon cancer and colon cancer organoids.

### EXAMPLE 10. GPR48 ANTIBODY HAS OBVIOUS INHIBITORY EFFECT ON GPR48 POSITIVE COLORECTAL CANCER CELL LINES

2-1H1 monoclonal antibody is used as the primary antibody to identify the expression of GPR48 in different colorectal cancer cell lines (SW480, HCT116, HT29 and P6C). Western blot analysis show that in these four cell lines, the expression of GPR48 in HT29 is lower, and the expression of GPR48 in SW480 is intermediate, and the expression of GPR48 in HCT116 and P6C cells is higher (FIG. 2). Thus, the two cell lines, HCT116 and P6C, can be used for the study of the relationship between an antibody against GPR48 and tumor therapy.

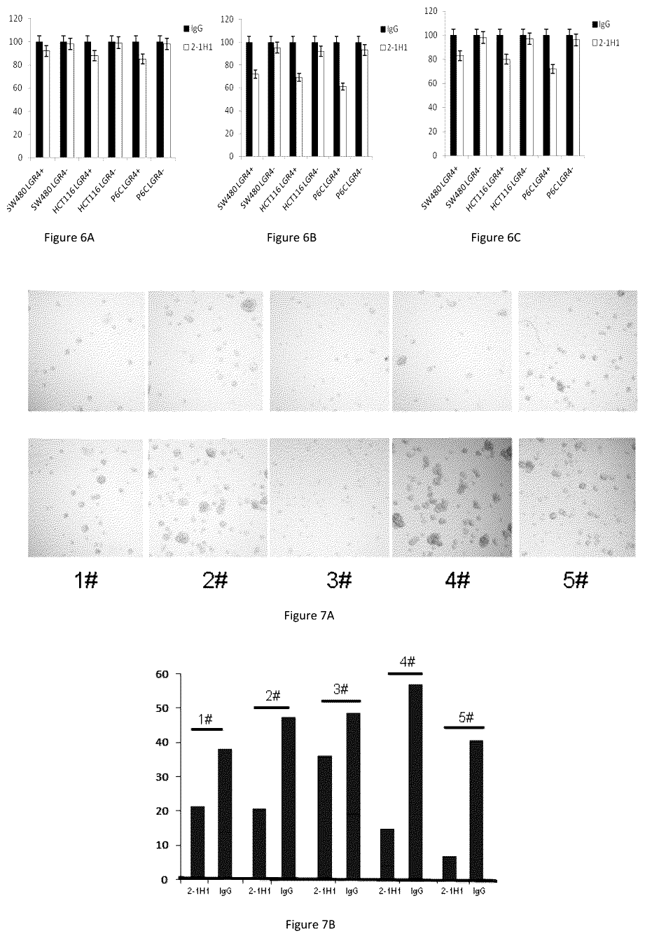

SW480, HCT116 and P6C cells are incubated with commercial GPR48 monoclonal primary antibody and fluorescently labeled secondary antibody. Cells expressing GRP48 (GPR48⁺) and cells not expressing GPR48 (GPR48⁻) are separated by flow cytometry. 2-1H1 monoclonal antibody at a final concentration of 10 μg/ml is added. Incubate with thiazolium blue (3-(4,5-dimethylthiazole-2)-2,5-diphenyltetrazolium bromide) for 24 hours, 48 hours and 72 hours respectively, and the optical density of OD value at 490 nm is measured with a microplate reader, to calculate cell viability. As shown in FIG. 6, 2-1H1 monoclonal antibody specifically inhibited the viability of GPR48 ⁺ cells.

Clone formation and tumorigenesis are the two most important criteria for testing the stemness of cancer stem cells. The changes in stemness of three GPR48 positive cells (SW480, HCT116 and P6C) and the control negative cell MCF7 after antibody treatment are determined by clonogenic technique. The specific steps of the clone formation method are as follows: digest the cells into single cells, centrifuge and discard the culture medium after the digestion is terminated, wash with PBS twice, and re-suspend with the culture medium. Adjust the cell density to 1×10⁴/ml, take 10 μl (1×10² cells) of the cell suspension and add it to 2 ml culture medium. Add 2-1H1 monoclonal antibody at a final concentration of 10 μg/ml, mix well and add to one well of a 6-well plate, and repeat 3 wells for each sample. Fresh culture medium is replaced every 3 days. After 14 days, the cells are fixed, stained with R-250, observed under a microscope, photographed, and counted to count the number of clones. 10 μg/ml mouse IgG is used as a negative control, and the treatment time and frequency of changing culture medium are the same as those of 2-1H1 antibody group. The commercial GPR48 monoclonal antibody is used as the control group, and the antibody concentration and treatment method are the same as 2-1H1 antibody group.

By counting the monoclonals, it is found that compared with the IgG control group, the ability of cell clone formation is inhibited to varying degrees in all the experimental groups added with 2-1H1 monoclonal antibody. Especially in P6C and HCT116 cells with high expression level of GPR48, the inhibition rate of 2-1H1 antibody to colony formation reach 76.4% and 51.3%, respectively. For SW480 cells with low expression level of GPR48, 2-1H1 monoclonal antibody also inhibited the colony formation to some extent (43.5%). The commercial GPR48 monoclonal antibody has a weak inhibitory effect on the colony formation of the three types of cells, especially in the P6C and HCT116 cells with a high expression level of GPR48, the inhibition rate of the commercial GPR48 antibody on the colony formation is only 9.7% and 15.4%. The inhibition rate results are shown in Table 2.

TABLE 2
Inhibitory effect of 2-1H1 antibody on colony formation
of GPR48 positive colorectal cancer cells

| | Antibody | | |
|---|---|---|---|
| | | | number of clones/ |
| | | | inhibition rate |
| | number of clones/ | | commercial |
| | colony formation | number of clones/ | GPR48 |
| | IgG negative | inhibition rate | monoclonal |
| Cells | control | 2-1H1 antibody | antibody control |
| P6C | 72/100% | 17/76.4% | 65/9.7% |
| SW480 | 23/100% | 13/43.5% | 17/26.1% |
| HCT116 | 39/100% | 19/51.3% | 33/15.4% |

### EXAMPLE 11. INHIBITORY EFFECT OF GPR48 ANTIBODY ON TUMOR ORGANOIDS

Colorectal cancer organoids (same as step 1 in Example 7) are separated and digested with TrypLE Express, filtered with a 400-mesh cell sieve, washed twice with phosphate buffer, and counted. 100 cells are inoculated on Matrigel, and 10 μg/ml of 2-1H1 monoclonal antibody is added at the same time. The fresh culture solution containing 2-1H1 monoclonal antibody is replaced every 2 days, 10 μg/ml of IgG is used as the control, and each sample is repeated for 3 wells. After 20 days, observe the number and size of organoids under a microscope, take pictures, count, and count the number of tumor organoids.

As shown in FIG. 7, in tumor organoids from 5 patients, 2-1H1 antibody significantly inhibited the formation rate and size of organoids. It can be seen from the figure that 2-1H1 antibody inhibited the tumor organoids of the five patients to varying degrees (inhibition rates ranged from 45% to 78%). Especially in patient No. 4 with the highest rate of organoid formation, the inhibitory effect is the most significant, and the inhibition rate reach 75%, indicating that 2-1H1 antibody has a high biological activity.

### EXAMPLE 12. GPR48 ANTIBODY HAS A GOOD THERAPEUTIC EFFECT ON MOUSE CECUM TUMOR

IgG and monoclonal antibody 2-1H1 are dissolved in PBS buffer and injected intraperitoneally into wild-type C57 mice at a dose of 10 mg/kg body weight once a week. After 6 weeks, the mice are weighed, dissected, and the main organs of the liver and spleen are observed. It is found that there is no obvious difference between the two groups. There is no obvious difference in the color and size of the liver and spleen. The average weight of the mice is basically the same, all at about 29 g. The mouse heart, liver, lung, small intestine, large intestine and other organs are respectively taken, fixed in formalin, embedded in paraffin, and stained with H&E. It is found that there is no significant change in the organ tissue structure of the mice, indicating that the antibody of the present invention has low toxicity to normal mice and has good biological safety.

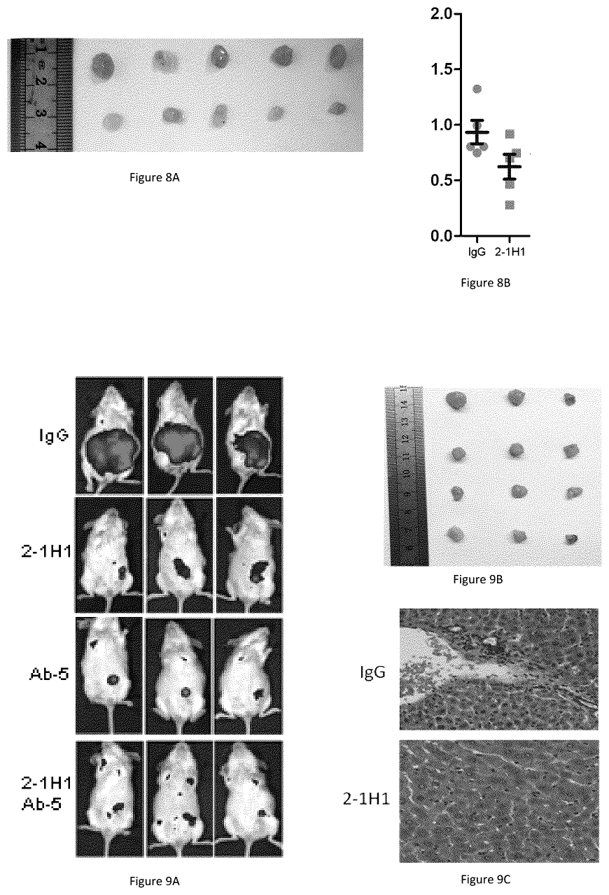

In situ inoculation of 10 tumor organoids (“colorectal cancer organoids” in step 1 of Example 7) in the cecum of NOD/SCID mice, the mice are dissected 3 weeks later, tumor formation is visible to the naked eye, indicating a mouse cecal tumor model is created successfully. The NOD/SCID mice inoculated with tumor organoids for 3 weeks are randomly divided into IgG control group and 2-1H1 monoclonal antibody experimental group, and intraperitoneal injection of antibody treatment experiment (IgG and 2-1H1 monoclonal antibody are dissolved in PBS buffer, injected once a week, and the dose is 10 mg/kg body weight). As shown in FIG. 8, after 5 weeks of administration, that is, 8 weeks after tumor inoculation, through dissection observation and tumor stripping and weighing, we found that the tumors of the mice in the antibody treatment group are significantly smaller than those in the IgG control group. It shows that 2-1H1 monoclonal antibody of the present invention has a good therapeutic effect on mouse cecal tumors.

### EXAMPLE 13. COMBINATION TREATMENT OF 2-1H1 AND PrPc MONOCLONAL ANTIBODY Ab-5 CAN BETTER INHIBIT THE GROWTH AND METASTASIS OF TUMOR IN SITU

Inoculate 2×10⁶ DeRed-P6C cells on the intestinal wall of the cecum of nude mice. The preparation method of DeRed-P6C cells with fluorescent marker groups is as follows: P6C cells are seeded in 6-well plates, and when the cells grow to 80-90% full, transfect the pDsRed2-N1 vector with lipofectemin2000 kit, change the medium (HDMEM containing 10% FBS) after 5 hours, culture routinely for 24 hours, digest the cells, make a single cell suspension, use flow cytometry to sort DeRed positive cells, the excitation light is 556 nm, the emission light is 586 nm, and then the DsRed positive cells are expanded and cultured, and continuously sorted for more than 10 times. When the proportion of DsRed positive cell is stable above 95%, the DsRed-P6C stable cell line is obtained. In the third week after the inoculation of DsRed positive cells, the in vivo imaging technology of small animals is used to observe whether the tumor inoculation is successful, and it is found that most of the tumor inoculation is successful.

Animal experiments of combination therapy are divided into 4 groups: \textcircled{1} IgG Cont 10 mg/kg, \textcircled{2} GPR48 2-1H1 10 mg/kg, \textcircled{3} PrPc Ab-5 10 mg/kg, \textcircled{4} combination: GPR48 2-1H1+PrPc Ab-5 each 10 mg/kg. 10 mice are present in each group. Antibody injection is started 3 weeks after tumor inoculation. After 8 weeks of treatment, obvious ascites had begun to appear in the IgG control group. The fluorescence information of abdominal tumors in mice is collected with an in vivo imager, and the fluorescence data of isolated organs are collected after dissection. After 8 weeks of treatment, the fluorescence of abdominal tumors in mice show that the GPR48 2-1H1 monoclonal antibody experimental group and the GPR48 2-1H1+PrPc Ab-5 combination treatment group are significantly lower than those in the IgG control group (P<0.01). Mice are dissected to obtain subcutaneous tumors. By measuring the tumor volume, we found that the tumor volume of the 2-1H1 treatment group is significantly lower than that of the IgG control group, and the combination treatment of GPR48 2-1H1+PrPc Ab-5 had a more significant inhibitory effect on the tumor (FIG. 9). The mouse liver is taken, fixed in formalin and embedded in paraffin, and H&E staining show that combination treatment significantly reduced the metastasis of tumor cells to the liver. The above results show that 2-1H1 antibody of the present invention can better inhibit tumor growth and metastasis when combined with the PrPc monoclonal antibody Ab-5.

### EXAMPLE 14. 2-1H1 INHIBITS Wnt SIGNALING PATHWAY IN TUMOR CELLS

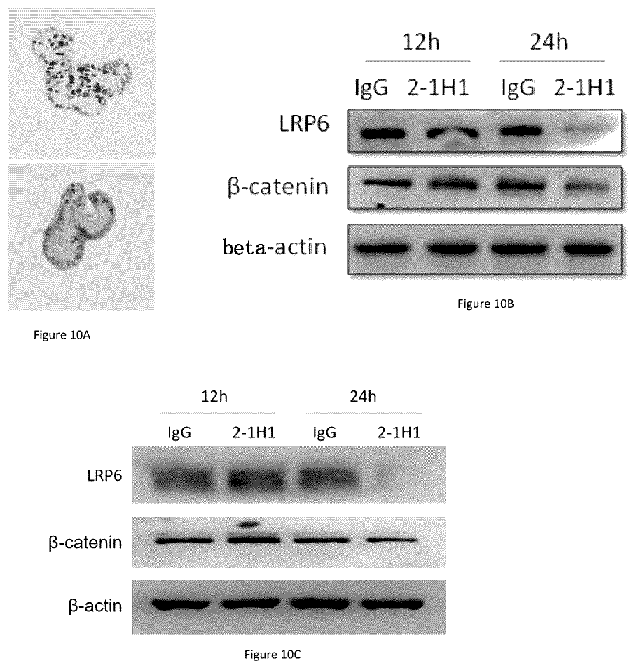

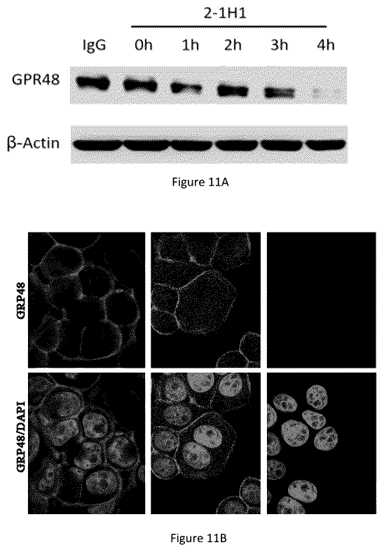

In order to further verify the specific mechanism of 2-1H1 antibody's inhibitory effect on the growth of GPR48 positive cells, we detected the expression of Ki67, a factor related to cell proliferation, in tumor organoids (“colorectal cancer organoids” in step 1 of Example 7), it is found that the protein is expressed at a higher level in tumor cells. GPR48 positive tumor cells in colorectal cancer organoids are sorted by flow cytometry, and GPR48 positive tumor cells are cultured into tumor organoids in an organoid culture system. 2-1H1 monoclonal antibody (concentration: 10 μg/ml) is added to the culture system. After immunohistochemical detection, it is found that 2-1H1 decreased the expression level of Ki67 protein compared with the IgG control group (A in FIG. 10). LRP6 is a receptor of the Wnt signaling pathway and conducts the downstream signaling pathway of Wnt. Western blot detection show that under the action of 10 μg/ml of 2-1H1, LRP6 decreased at 12 hours, and the key protein β-catenin of WNT signaling pathway decreased significantly at 24 hours, indicating that 2-1H1 monoclonal antibody inhibits WNT signaling pathway (B in FIG. 10). This phenomenon explains why GPR48 positive cell colony-forming ability decreased, the incidence of tumor organoids decreased, and tumor growth is inhibited in nude mice after 2-1H1 treatment. The same result is also verified in the GPR48 positive tumor cell line HCT116 cells (C in FIG. 10).

### EXAMPLE 15. 2-1H1 MONOCLONAL ANTIBODY CAUSES DEGRADATION OF ANTIGEN GPR48

In order to verify the neutralizing activity of 2-1H1 monoclonal antibody on the antigen, we treated the tumor cell line P6C with 2-1H1 at a final concentration of 10 μg/ml, and the expression level of GPR48 is detected at 0, 1, 2, 3, and 4 hours respectively using western blotting. It is found that 2-1H1 significantly reduced the expression of GPR48 in tumor cells at 4 hours (A in FIG. 11, IgG treated for 4 hours as a negative control). To verify this result, we conjugated the green fluorescent protein GFP to 2-1H1 (routine biological methods). Incubate tumor cell P6C with 2-1H1-GFP at 4 ° C. for 30 minutes, wash off the unbound 2-1H1-GFP antibody with 4 ° C. pre-cooled PBS, and immediately fix the control group with -20° C. formaldehyde, and in the experimental group (1), 37° C. cell culture medium containing 10 μg/ml 2-1H1 final concentration is added, in experimental group (2), 37° C. cell culture medium containing commercial GPR48 monoclonal antibody (R&D) at a final concentration of 10 μg/ml is added. After 15 minutes, the cells are fixed with formaldehyde at −20° C., the nuclei are stained with DAPI, and the fluorescent signal of GFP is observed under a confocal microscope. The results show that GPR48 in the control group is localized on the cell membrane, and 2-1H1 incubated the cells for 15 minutes, which significantly promoted the degradation of GPR48 (B in FIG. 11, the left panel is the control group, the middle panel is the commercial GPR48 monoclonal antibody incubation group, and the right panel is the 2-1H1 incubation group), indicating that 2-1H1 has neutralizing activity to degrade antigen GPR48.

The present invention has been described in detail above. For those skilled in the art, without departing from the spirit and scope of the present invention, and without unnecessary experiments, the present invention can be practiced in a wider range under equivalent parameters, concentrations and conditions. While specific examples of the invention have been shown, it should be understood that the invention can be further modified. In a word, according to the principles of the present invention, this application intends to include any changes, uses or improvements to the present invention, including changes made by using conventional techniques known in the art and departing from the disclosed scope of this application. Applications of some of the essential features are possible within the scope of the appended claims below.

### INDUSTRIAL APPLICATION

The present invention designs and synthesizes G protein coupled receptor 48 antigen peptides, immunizes Balb/c mice, and undergoes fusion, clonal screening, preparation of ascites, and purification of ascites by affinity chromatography to obtain a hybridoma cell 1H1 that can secrete a monoclonal antibody specifically recognizing human G protein coupled receptor 48. The monoclonal antibody 2-1H1 secreted by 1H1 not only has strong specificity and sensitivity to QLPEDAFKNFPFLEE peptide, but also has strong specificity and sensitivity to GPR48 protein in human tissues and cells. The monoclonal antibody can be applied to immunological experimental techniques such as western blotting, immunofluorescence and immunohistochemistry; it can be applied to flow cytometry experimental techniques such as flow cytometry analysis and flow cytometry sorting; it can be applied to the scientific research of signaling pathways such as subcellular localization and protein interaction. Monoclonal antibody 2-1H1 can be used for clinical drug development of human GPR48 positive tumors such as colorectal cancer.

1-36. (canceled)

37. An antibody, comprising a heavy chain variable region comprising amino acid sequence HCDR1, amino acid sequence HCDR2, and amino acid sequence HCDR3, wherein
the amino acid sequence HCDR1 comprises positions 26-33 of SEQ ID NO. 2,
the amino acid sequence HCDR2 comprises positions 51-60 of SEQ ID NO. 2, and
the amino acid sequence HCDR3 comprises positions 99-108 of SEQ ID NO. 2.

38. The antibody according to claim 37, further comprising a light chain variable region comprising amino acid sequences LCDR1, LCDR2, and LCDR3 wherein
the amino acid sequence LCDR1 comprises positions 27-32 of SEQ ID NO. 1,
the amino acid sequence LCDR2 comprises positions 50-52 of SEQ ID NO. 1,
the amino acid sequence LCDR3 comprises positions 89-97 of SEQ ID NO. 1, and
wherein the light chain variable region comprises an amino acid sequence having an identity of at least 75% to SEQ ID NO. 1.

39. The antibody according to claim 37, wherein the heavy chain variable region comprises an amino acid sequence having an identity of at least 75% to SEQ ID NO. 2.

40. The antibody according to claim 37, wherein the antibody is a full-length antibody, a Fab fragment, a F(ab′)2 fragment, a murine antibody, or a single-chain Fv fragment.

41. The antibody according to claim 40, wherein the heavy chain constant region of the full-length antibody is an IgG1 subtype.

42. The antibody according to claim 40, wherein the light chain constant region of the full-length antibody is a λ subtype.

43. Any of the following materials:
(A1) A mouse hybridoma cell line 1H1, deposited in the General Microbiology Center of China Committee for the Collection of Microorganisms as CGMCC No. 20290;
(A2) A monoclonal antibody, secreted by the mouse hybridoma cell line 1H1 in (A1);
(A3) An antibody obtained by immunizing an animal with a conjugate of the polypeptide shown in SEQ ID NO. 5 and a carrier protein as an immunogen;
(A4) A humanized antibody obtained by humanizing the antibody, or the monoclonal antibody of (A2), or the antibody of (A3);
(A5) An active fragment of the antibody, being any one of a single chain antibody, a Fab region of an antibody, an antigen-binding fragment, derived from the antibody or the monoclonal antibody of (A3) or the antibody of (A4) or the humanized antibody in (A5);
(A6) A nucleic acid molecule encoding the antibody, the monoclonal antibody of (A2), or the antibody of (A3) or the humanized antibody in (A4) or an active fragment of the antibody in (A5);
(A7) An expression cassette, a recombinant vector, a recombinant cell or a recombinant bacteria containing the nucleic acid molecules in (A6);
(A8) A kit containing the antibody, or the monoclonal antibody of (A2), the antibody of (A3), the humanized antibody in (A4), the active fragment of the antibody in (A5), the nucleic acid molecule in (A6), or the expression cassette, the recombinant vector, the recombinant cell or the recombinant bacteria in (A7);
(A9) A polypeptide shown in SEQ ID NO. 5;
(A10) A conjugate of the polypeptide shown in SEQ ID NO. 5 and a carrier protein; and
(A11) A pharmaceutical composition, characterized in that: the pharmaceutical composition comprises the antibody, the monoclonal antibody in (A2), the antibody in (A3), the humanized antibody in (A4) or the active fragment of the antibody in (A5).

44. The material according to claim 43, characterized in that: in the nucleic acid molecule in (A6), the nucleotide sequences encoding LCDR1, LCDR2 and LHCDR3 in the light chain variable region of the antibody are as shown in positions 79-96, 148-156, and 265-291 of SEQ ID NO. 3 from the 5′ end;
preferably, in the nucleic acid molecule in (A6), the nucleotide sequence encoding the light chain variable region of the antibody is shown in SEQ ID NO. 3 or has an identity of 99% or more, 95% or more, 90% or more, 85% or more, 80% or more or 75% or more to SEQ ID NO. 3.

45. The material according to claim 43, characterized in that: in the nucleic acid molecule in (A6), the nucleotide sequences encoding HCDR1, HCDR2 and HCDR3 in the heavy chain variable region of the antibody are shown in positions 76-99, 151-180, and 295-324 of SEQ ID NO. 4 from the 5′ end; and
preferably, in the nucleic acid molecule in (A6), the nucleotide sequence encoding the heavy chain variable region of the antibody is SEQ ID NO. 4 or has an identity of 99% or more, 95% or more, 90% or more, 85% or more, 80% or more or 75% or more to SEQ ID NO. 4.

46. The material according to claim 43, characterized in that: the pharmaceutical composition in (A1) also contains an anti-tumor drug.

47. The material according to claim 46, characterized in that: the anti-tumor drug is a cellular prion protein PrPc monoclonal antibody Ab-5.

48. The material according to claim 47, characterized in that: in the anti-tumor drug, the mass ratio of the monoclonal antibody in (A2) to the cellular prion protein PrPc monoclonal antibody Ab-5 is 1:1.

49. Any of the following methods:
(1) A method for preparing the antibody according to claim 37 or the monoclonal antibody in (A2) or the antibody in (A3) or the humanized antibody in (A4) or the active fragment of the antibody in (A5) or the kit in (A8);
(2) A method for preparing the pharmaceutical composition in (A11) or the material;
(3) An anti-tumor method;
(4) A method for inhibiting tumor growth;
(5) A method for inhibiting tumor metastasis;
(6) A method for inhibiting the growth of tumor organoids;
(7) A method for inhibiting tumor cell clone formation;
(8) A method for inhibiting GPR48;
(9) A method for detecting GPR48;
(10) A method for inhibiting the viability of GPR48-positive cells;
(11) A method for inhibiting WNT signaling pathway;
(12) A method for inhibiting the expression of Ki67 protein, LRP6 protein and/or β-catenin protein; and
(13) A method for preparing synergistic auxiliary preparations of an anti-tumor drug.

50. The method according to claim 49, characterized that in method (1), the nucleic acid molecule in (A6) or the expression cassette, the recombinant vector, the recombinant cell or the recombinant bacterium in (A7) or the peptide in (A9), or the conjugate in (A10) is used.

51. The method according to claim 49, characterized that in method (2) any one of the following is used:
A: the nucleic acid molecule in (A6) or the expression cassette, the recombinant vector, the recombinant cell or the recombinant bacterium in (A7) or the peptide in (A9), or the conjugate in (A10); and
B: the antibody, the mouse hybridoma cell line 1H1 in (Al), the monoclonal antibody in (A2), the antibody in (A3), or the humanized antibody in (A4) or the active fragment of the antibody in (A5).

52. The method according to claim 49, characterized that in all methods (3) to (17) the antibody.

53. The method according to claim 49, characterized that in method (13) the antibody, the monoclonal antibody in (A2), the antibody in (A3), or the humanized antibody in (A4) or the active fragments of the antibody in (A5) is used.

54. The method according to claim 49, characterized in that the anti-tumor drug is Ab-5, a monoclonal antibody against cellular prion protein PrPc.

55. The method according to claim 49, characterized in that: the tumor is a GPR48 positive solid tumor; the tumor organoid is a GPR48 positive solid tumor organoid; and the tumor cell is a GPR48 positive tumor cell.

56. The method according to claim 55, characterized in that: the solid tumor is selected from any of the following: colorectal cancer, breast cancer, prostate cancer, pancreatic cancer, gastric cancer;
the tumor cell is colorectal cancer cells SW480, HCT116, HT29 or P6C; and
the tumor organoid is a colorectal cancer organoid.
